(19) United States
(12) Patent Application Publication
(10) Pub. No.: US 2014/0341999 A1
(43) Pub. Date: Nov. 20, 2014

(54) ANTITUBERCULAR COMPOSITION AND METHOD FOR PRODUCING SAME

(71) Applicant: OBSCHESTVO S OGRANICHENNOY ONVETSTVENNOSTYU ''IBMKH-EKOBIOFARM', Moscow (RU)

(72) Inventors: Olga Mikhaylovna Ipatova, Moscow (RU); Natalya Velorikovna Medvedeva, Moscow (RU); Vladimir Nikolayevich Prozorovskiy, Moscow (RU); Maksim Aleksandrovich Sanzhakov, Moskovskaya obl. (RU); Yelena Georgiyevna Tikhonova, Moscow (RU); Oksana Sergeevna Druzhilovskaya, Moskow (RU)

(73) Assignee: OBSCHESTVO S OGRANICHENNOY ONVETSTVENNOSTYU ''IBMKH-EKOBIOFARM', Moscow (RU)

(21) Appl. No.: 14/361,753

(22) PCT Filed: Nov. 15, 2012
(86) PCT No.: PCT/RU2012/000936

§ 371 Date: May 30, 2014

(30) Foreign Application Priority Data
Dec. 6, 2011 (RU) 2011149543

(51) Int. Cl. A61K 47/12; A61K 31/395; A61K 47/26; A61K 31/435; A61K 31/496; A61K 47/24; A61K 9/14; A61K 31/44

(52) U.S. Cl. CPC A61K 47/12; A61K 9/14; A61K 31/395; A61K 31/44; A61K 31/435; A61K 31/496; A61K 47/24; A61K 47/26
USPC 424/489; 514/468; 514/357; 514/278; 514/254.11

(57) ABSTRACT

The invention relates to the field of pharmaceutics and medicine and concerns a medicinal composition of antitubercular preparations with a phospholipid transport system, the composition consisting of a fatty acid salt, phosphatidylcholine of vegetative origin (73-97%), maltose and an antitubercular agent selected from rifamycin, protionamide, rifabutin and rifapentine, and a method for producing the composition.

The present invention relates to the field of medicine and pharmacology and concerns a medicinal composition of antitubercular preparations with a phospholipid transport system, and a method for producing the same.

At present, antitubercular preparations are divided into basic and reserve ones. The basic preparations comprise isoniazid, rifampycin, pyrazinamide, streptomycin and ethambutol.

The reserve preparations comprise ethionamide, prothionamide, antibiotics like cycloserine, capreomycin, kanamycin, amikacin, rifabutin, para-aminosalicylic acid (PASA); the same group comprises fluorchinilones like lomefloxacin, ciprofloxacin and oflaxocin.

The basic preparations, in particular under the form of combinations, are mainly used in the case of the first-time detection of tuberculosis (usually, a combination of isoniazid and rifampycin with other basic preparations is prescribed). They have resort to reserve preparations in the case of insufficient efficiency of previous chemotherapy, development of mycobacterial resistance, bad endurance for previously administered preparations.

The basic preparations are highly efficient but the mycobacterial resistance to the same develops rather quickly (3-4 months). The resistance grows much more slowly in the case of a simultaneous use of different preparations. That is why the main concept of the modern tuberculosis treatment is the combined two-stage chemotherapy.

Moreover, it is necessary to take into consideration that the effect of antitubercular preparations is usually accompanied by side effects the intensity of which can grow at their joint application.

Protionamide makes part of reserve preparations. It is quickly absorbable from the gastrointestinal tract (GIT), Cₘₐₓ being of 2-3 hours; it easily penetrates into tissues, is partly metabolized and is eliminated with urine and feces.

Rifabutin (Mycobutin) is a reserve preparation. It is a semisynthetic antibiotic and is efficient against different kinds of mycobacteria, including mycobacteria of tuberculosis. It is practically water-insoluble.

It is quickly and completely absorbed from the GIT, Cₘₐₓ being of 2-4 hours. T½ is 35-40 hours.

It penetrates well into the organs and tissues (the highest concentration being in the lungs).

Rifampycin makes part of basic preparations. It is a semisynthetic antibiotic of a wide spectrum of action. It is active against the mycobacteria of tuberculosis and leprosy, it has effect on gram-positive bacteria (in particular staphylococci) and on gram-negative bacteria (meningococci, gonococci) as well as on brucellosis, legionellosis, classical typhus and trachoma pathogens. It is efficient when administered orally and has a wide range of the antibacterial effect.

It is well and quickly absorbed in GIT, Cₘₐₓ being of 2-2.5 hours at oral administration. It easily penetrates into tissues and liquids of the body and is detected in therapeutic concentrations in pleural exudation, in expectoration, in the cavern content, in osseous tissues; a small concentration grows in liver and kidney tissues; it is eliminated with bile and urine.

Rifapentin (Rifapex) is an antibiotic from the class of rifamycins, providing for a higher inhibiting effect against Mycobacterium tuberculosis and having a longer half time of elimination, compared to rifampycin, a preparation from the basic group of tuberculosis therapy (TB).

Simultaneously with Rifapentin, reserve preparations are prescribed, such as isoniazid, ethambutol, pyrasinamide. Rifapentin is one of the most promising preparations, the regime of a combined treatment on the basis of rifapentin can provide an important clinical advantage for patents with TB, sensitive to medicinal therapy, enabling to shorten the treatment course.

All the above mentioned preparations are used in medicinal forms for oral administration (capsules, tablets or dragees), and only rifampycin is used as well under the form for injections.

In general all the antitubercular preparations represent poorly soluble compositions with a limited biological availability. The use of these preparation needs long periods with high dosages, mostly in combined treatment procedures due to the medicinal resistance and generates grounds for searching for new medicinal preparations and for improving known ones (M. D. Mashkovsky, Medicinal preparations, ed. 15, Moscow, Novaya Volna, 2006, p. 857-871 [1]; J. van den Boogaard, G. S. Kibiki, E. R. Kisanga, M. J. Boeree, and R. E. Aarnoutse. New Drugs against Tuberculosis: Problems, Progress, and Evaluation of Agents in Clinical Development.//Antimicrob. Agents Chemother.—2009.—53(3)—p. 849-862 [2]).

An original technology is known enabling to obtain a phospholipid transport nano-system with a particle size of less than 30 nm, and disclosing a basic possibility to get medicinal preparations where the active substance is provided with a transport nano-system on the basis of vegetative phospholipids (patent RU No 2391966, Cl. 20.06.2010 [3]). It is shown that as a result of such incorporation, the pharmacokinetics of medicinal substances is modified while administered into the organism, their bioavailability and the therapeutic efficiency are increased.

The presence of a hydrocarbonic area in phospholipids structures, including in mycella and liposomes, enables hydrophobic compounds to be included into the same. This principle can be used as a base for including a series of medicinal substances into phospholipid nano-particles, such substances presenting amphipathic, lypophilic and hydrophobic properties.

There is a known nano-system, stable in storage, with the particle size of 10 to 30 nm, comprising phosphatidylcholine and maltose, designed to incorporate medicinal preparations into the phospholipid nano-particles [3].

Thanks to its chemical structure, said phospholipid transport nano-system is able to serve as a carrier of (hydrophobic) bioactive preparations, either soluble or insoluble in biological liquids, but, unfortunately, the medicinal substances are far from being all able to be incorporated into the transport system obtained on the basis of phosphatidylcholine, due to their physico-chemical properties and their chemical structure.

The aim of the present invention is to develop an antitubercular composition on the basis of medicinal substances presenting poor solubility, low bioavailability, pronounced side effects, the composition being free from said drawbacks.

According to the present invention, an antitubercular composition in the form of nano-particles with a size of 20 to 80 nm is disclosed, comprising a fatty acid salt, phosphatidylcholine of vegetative origin (73-97%), maltose and an antitubercular agent selected from rifamycin, protionamide, rifabutin and rifapentine, with the following ratio of the components, mass %:

| | |
|---|---|
| Phosphatidylcholine | 19-25; |
| Fatty acid salt | 1.5-10; |
| Maltose | 67-78; |
| Antitubercular agent | 1-10. |

As a fatty acid salt, preferably sodium oleate is used.

It is described as well a method for producing said antitubercular composition consisting in the fact that a fatty acid salt, maltose and an antitubercular agent are emulsified in water, phosphatidylcholine is added and the suspension obtained is submitted to 1 to 10 homogenizing cycles under a high pressure of 800-1500 bars at a temperature of 40-45° C., followed by lyophilization.

### EXAMPLES

### Obtaining Compositions of Antitubercular Preparations Built-in Into Nano-Particles of Phospholipids and Sodium Oleate

1.0 g of maltose monohydrate is dissolved in 150 ml of deinonized water. To the solution obtained, phospholipid is added and stirred with a blender to homogeneity. The volume of the solution is brought with water to 200 ml. Under constant stirring, 0.02 g of rifampycin substance and 0.02 g of sodium oleate are successively added in the dry state.

The crude emulsion obtained is processed with ultrasound on an ultrasonic bath for 1 min, followed by heating to 42-45° C.

After ultrasonic processing, the crude emulsion is homogenized on a microfluidizer for 5 min (7 cycles of 0.7 min) at a pressure of 1000 atm; the temperature of the product at the outlet is maintained within 40 to 45° C. The pH of the nano-emulsion obtained after the microfluidization is brought to the value 7.3-7.4 with the 1M solution of HCl. After that, the product is filtered through a preliminary glass fiber filter and through a membrane filter with the pores size of 0.22 μm. The filtrate is poured into vials of 10 ml. The product is dried by lyophilization.

The lyophilized powder is dissolved (rehydrated) in 10.0 ml of distilled water. The main physico-chemical characteristics of the preparation are determined: light transmission at the wavelength 660 nm, average size of particles as to the polymodal distribution, content of the built-in substance and pH.

In a similar way, compositions on the base of protionamide, rifabutin and rifapentin are obtained (Table 1).

TABLE 1

| | Loaded amount, g | | | | Obtained | |
|---|---|---|---|---|---|---|
| Name of the active substance/ | Active | | Sodium | | Built-in active | Particle |
| manufacturer | substance | Phospholipides | oleate | Maltose | substance, mg | size, nm |
| Rifampycin/Sandoz Pvt.Ltd. | 0.02 | 0.25 | 0.02 | 1.0 | 14.3 | 21.4 |
| Protionamide/Guangxi Gulin | 0.02 | 0.25 | 0.02 | 1.0 | 13.4 | 42.0 |
| Pharm.Fact. | | | | | | |
| Rifabutin/Sandoz Pvt.Ltd. | 0.02 | 0.25 | 0.02 | 1.0 | 19.2 | 47.1 |
| Rifapentin/Nanjing Foreign | 0.02 | 0.25 | 0.02 | 1.0 | 19.0 | 39.1 |
| Economic&Trade Development Co., | | | | | | |
| LTD. | | | | | | |

The preparations obtained according to the above described methods are water-soluble, can be used perorally, and when observing the aseptic conditions of their obtaining, intravenously.

The use of sodium oleate makes the phospholipid system not only more universal to be built-in but produces an additional therapeutic antitubercular effect (MINAMI K. Bactericidal action of oleic acid for tubercle bacilli.//Bacteriol.—1957.—Vol. 73, No3.—P. 338-352) [3].

The efficiency of protionamide and rifampycin built-in into liposomes was studied, under the conditions of monotherapy for an experimental generalized tuberculosis in mice, induced by a medicine-sensitive strain M. tuberculosis Erdman. The substances to test were IG and S/c administered with dosages corresponding to the starting substances of protionamide (12.5 mg/kg) or rifampycin (10 mg/kg). Treatment in all the experimental groups was started on the third day after contamination. The treatment course period was 6 weeks.

Protective and therapeutic properties of antitubercular preparations included into the phospholipid nano-particles were shown up, which were documented by the absence of experimental animal death during the whole treatment period and by the gravity reduction of the course of an experimental generalized tuberculosis in mice. As a result, the average total affection index after the application of protionamide was reduced to 55, 43 and 55, 52 (respectively, in the S/c and IG administration) against 62, 31 in the case of the initial substance application. In the treatment of contaminated mice with rifampycin built-in into phospholipid nano-particles, the average total affection index for the S/c way of injection was of 8.6, and for the IG administration, it was of 1.60 (for the initial substance, it was 9.06). It was established the curative effect dependence of the way of administering the substances under study: protionamide built-in into nano-particles showed the most distinct effect in the S/c injection, and rifampycin in the IG administration. In the optimal way of administration, the treatment efficiency growth was of +13.62% for protionamide built-in into nano-particles, and of +34.66% for rifampycin.

The experimental studies performed to determine the acute toxicity of the preparations protionamide, rifampycin and rifabutin designed on the basis of a transport system showed that these preparations were little toxic and did not demonstrate any negative effect on the internal organs under intravenous injection.

The results of toxicometry, the data of observing the experimental animals for 14 days after the acute administration, as well as the necropsy data enabled to attribute:
a rifampycin preparation built-in into phospholipid nano-particles and used under intravenous injection, to the classes IV-V of little toxic and practically nontoxic substances;
a protionamide preparation on the base of nano-particles in intravenous injection, to the class V of practically nontoxic substances;
a rifabutin preparation built-in into phospholipid nano-particles and used under intravenous injection, to the classes IV-V of little toxic and practically nontoxic medicinal substances.

Studies of acute toxicity of the Rifampycin preparation built-in into phospholipid nano-particles were carried out in comparison with the similar preparation “Rifampycin-Ferein®, a lyophilisation product to make a solution for injections of 0.15 g” manufactured by the Closed Joint Stock Venture “Bryntsalov-A”. It was shown that the rifampycin preparation based on nano-particles, is 25-30% less toxic than the registered similar preparation (Table 2).

### Acute Toxicity in Intravenous Injection

TABLE 2

| | | Toxicity | LD₅₀ | LD₁₆ | LD₈₄ |
|---|---|---|---|---|---|
| | | degree after | mg/kg | mg/kg | mg/kg |
| No | Preparation name | Hodge Sterner | Males/females | | |
| 1. | Rifampycin | IV-V | 710 ± 50/ | 550 ± 30/ | 900 ± 90/ |
| | | | 710 ± 50 | 550 ± 30 | 900 ± 90 |
| 2. | Rifampycin-Ferein ® | | 590 ± 40/ | 430 ± 30/ | 710 ± 80/ |
| | | | 530 ± 30 | 400 ± 20 | 680 ± 60 |
| 3. | Protionamide | V | 1263 ± 55/ | 1085 ± 64/ | 1471 ± 91/ |
| | | | 1198 ± 33 | 996 ± 30 | 1442 ± 38 |
| 4. | Rifabutin | IV-V | 750 ± 50/ | 630 ± 60/ | 910 ± 100/ |
| | | | 710 ± 40 | 540 ± 50 | 870 ± 70 |

A comparative experimental study of the general toxicity and of the irritating properties of the preparations rifampycin, protionamide and rifabutin, built-in into phospholipids nano-particles was carried out as well, in comparison with the respective substances in intragastric administration.

The results obtained on the basis of the toxinometry data, for observing experimental animals for 14 days after the acute administration, as well as the data of necropsy are given in Table 3.

### Acute Toxicity in Intragastric Administration

TABLE 3

| | | Toxicity | | | | |
|---|---|---|---|---|---|---|
| | | degree after | LD₅₀ mg/kg | | | |
| | | Hodge | preparation | substance | preparation | substance |
| No | Preparation name | Sterner | mice | | rats | |
| 1. | Rifampycin | IV-V | 3650 ± 80 | 1700 ± 50 | 5000 ± 300 | 2425 ± 175 |
| 2. | Protionamide | V | 5100 ± 150 | 2850 ± 100 | 8100 ± 200 | 6800 ± 150 |
| 3. | Rifabutin | IV-V | 2750 ± 80 | 1700 ± 100 | 2500 ± 60 | 1650 ± 50 |

The results obtained in the study of the subacute toxicity for the preparation rifampycin provided with a transport system, at its intragastic administration once a day for 21 days at dosages of 20 and 600 mg/kg in rats of both sexes, compared to the substance at the dosages of 10 and 300 mg/kg showed that both preparations under study did not lead to any noticeable changes in the vital indices of experimental animals. Pathomorphism investigations did not reveal any modifications in the internal organs, demonstrating a negative adverse effect.

The study of subacute toxicity for the preparation and the substance in their intragastric administration once a day for 21 days at equitoxic dosages in rats of both sexes showed that both preparations under study do not induce any noticeable changes in the biochemical indices of blood. Pathomorphism investigations did not reveal any modifications in the internal organs either, that could demonstrate a negative adverse effect of the preparations. According to the results of morphological and histological studies, the intragastric administration of the preparations under study did not have any irritating effect on the esophagus, stomach or intestine.

The results obtained in the study of subacute toxicity for the preparation rifabutin provided with a transport system, at its intragastic administration once a day for 21 days at dosages of 40 and 1000 mg/kg in rats of both sexes, compared to the substance at dosages of 20 and 500 mg/kg showed that both preparations under study do not induce any noticeable changes in the vital indices of the experimental animals. Pathomorphism investigations did not reveal any modifications in the internal organs either, that could demonstrate a negative adverse effect.

The revealed negative trends in the effect of the preparations under study in a maximal dosage (oppression of erythro- and leucopoiesis, increase in blood coagulation time, increase of the arterial pressure level, increase of the central nervous system excitability) were of a reversible character.

The values of the physiological indices modifications due to the use of the preparations rifampycin, protionamid and rifabutin incorporated into a phospholipid transport system were compared to analogous modifications in the case of use of corresponding substances. In all the trials, the degree of indices modifications induced by both preparations in equitoxic dosages was practically the same: no cases of a reliable difference between the preparation and the substance were recorded.

At the same time, the revealed equivalence was observed in the use of rifampycin incorporated into phospholipid nano-particles with practical dosages two times superior to those for the substances.

The results obtained enable to state that the rifampycin preparation on the basis of phospholipid particles is almost 2 times less toxic than the substance.

The protionamid preparation incorporated into the phospholipid nano-particles is almost 1.2-1.7 times (depending on the kind of animals) less toxic than the substance.

The rifabutin preparation on the basis of phospholipid nano-particles is 1.5 times less toxic than the substance.

The results obtained enable to make a conclusion on the efficiency and the toxicity decrease in antitubercular preparations built-in into phospholipid nano-particles. Taking into consideration a high need of phthisiology to develop efficient methods of tubercular patients treatment, the problem of improving efficiency and reducing toxicity for antitubercular preparations is particularly pressing.

1. Antitubercular composition in the form of nano-particles with a size of 20 to 80 nm, comprising a fatty acid salt, phosphatidylcholine of vegetative origin (73-97%), maltose and an antitubercular agent selected from rifamycin, protionamide, rifabutin and rifapentine, with the following ratio of the components, mass %:

| | |
|---|---|
| Phosphatidylcholine | 19-25; |
| Fatty acid salt | 1.5-10; |
| Maltose | 67-78; |
| Antitubercular agent | 1-10. |

2. Composition of claim 1, wherein sodium oleate is used as a fatty acid salt.

3. Method for producing the antitubercular composition of claim 1, wherein a fatty acid salt, maltose and an antitubercular agent are emulsified in water, phosphatidylcholine is added and the suspension obtained is submitted to 1 to 10 homogenizing cycles under a high pressure of 800-1500 bars at a temperature of 40-45° C., followed by lyophilization.
